(19) United States
(12) Patent Application Publication
(10) Pub. No.: US 2019/0310225 A1
(43) Pub. Date: Oct. 10, 2019

(54) MICROFLUIDIC ORGANIC ELECTROCHEMICAL TRANSISTOR SENSORS FOR REAL TIME NITRIC OXIDE DETECTION

(71) Applicant: The Charles Stark Draper Laboratory, Inc., Cambridge, MA (US)

(72) Inventor: Hongmei Zhang, Lexington, MA (US)

(21) Appl. No.: 16/376,264

(22) Filed: Apr. 5, 2019

Related U.S. Application Data
(60) Provisional application No. 62/653,991, filed on Apr. 6, 2018.

(51) Int. Cl. G01N 27/414; G01N 33/543; H01L 51/00; G01N 33/00

(52) U.S. Cl. CPC G01N 27/4145; G01N 33/0037; H01L 51/0037; G01N 33/54393

(57) ABSTRACT

The present disclosure describes a solution to rapidly detect trace amounts of biomarkers present in a fluid sample. The solution can be, for example, used to diagnosis sepsis through the detection of nitric oxide. The solution includes one or more organic electrochemical transistors that are functionalized with a bio-recognition coating. The bio-recognition coating can bind or otherwise interact with the biomarkers to change the transconductance of the organic electrochemical transistors. The solution can detect the change in the transconductance of the organic electrochemical transistors and signal the presence of the biomarker.

### CROSS-REFERENCE TO RELATED APPLICATIONS

The present application claims the benefit, under 35 USC § 119(e), of the filing of U.S. Provisional Patent Application 62/653,991 filed Apr. 6, 2018. This provisional application is incorporated herein by reference for all purposes.

### BACKGROUND OF THE DISCLOSURE

Biomarkers can indicate the existence of biological conditions. For example, antibodies can be a biomarker indicating the existence of a bacterial infection. Biomarker detection and testing can be time consuming and labor intensive. Processes to detect biomarkers can have a low limit of detection (e.g., have a high sensitivity), but may take hours or days to perform. However, processes to rapidly detect biomarkers have a high limit of detection (e.g., have a low sensitivity).

### SUMMARY OF THE DISCLOSURE

The present disclosure describes systems capable of rapidly detecting biomarkers while maintaining a low limit of detection. The system can include organic electrochemical transistors (OECTs). The OECTs' channel material or gate electrode can be coated with or include a bio-recognition element. The bio-recognition coating is configured to bind with biomarkers. Binding of biomarkers to the bio-recognition coating induces gate voltage change, which results in a change in the source and drain current of the OECT. The magnitude in the current change can indicate the amount of biomarker present in a test sample. The OECTs can be incorporated into microfluidic devices to provide rapid, multiplexed detection of free biomarkers. The OECTs can also be incorporated into other testing and diagnostic devices such as well plates, sample holders, probes, or implantable devices.

According to at least one aspect of the disclosure, a microfluidic device can include a fluid chamber to hold a sample fluid. The device can include a biomarker sensor at least partially disposed in a wall defining the fluid chamber. The biomarker sensor can include a drain electrode and a source electrode. The biomarker sensor can include a channel material electrically coupling the drain electrode and the source electrode. The biomarker sensor can include a functional coating disposed on a surface of the channel material. The functional coating can bind with a biomarker in the sample fluid and, responsive to the binding of the biomarker with the functional coating, change a conductivity of the channel material.

In some implementations, the channel material can include poly(3,4-ethylenedioxythiophene) polystyrene sulfonate. The functional coating can include hemin. The biomarker can be nitric oxide. In some implementations, the functional coating can include an aptamer or an antibody.

In some implementations, the channel material between the drain electrode and the source electrode can have a length between 5 μm and 50 μm and a width between 25 μm and 125 μm. The device can include a microfluidic flow channel fluidically coupled with the fluid chamber. The microfluidic flow channel can include at least one separation region to remove undesirable particles from the fluid sample.

According to at least one aspect of the disclosure, a microfluidic device can include a fluid chamber to hold a sample fluid. The device can include a biomarker sensor at least partially disposed in a wall defining the fluid chamber. The biomarker sensor can include a drain electrode, a source electrode, and a gate electrode. The biomarker sensor can include a channel material electrically coupling the drain electrode and the source electrode. The biomarker sensor can include a functional coating disposed on a surface of the gate electrode. The functional coating can bind with a biomarker in the sample fluid and, responsive to the binding of the biomarker with the functional coating, change a gate voltage of the gate electrode.

In some implementations, the channel material can include poly(3,4-ethylenedioxythiophene) polystyrene sulfonate. The functional coating can include hemin. The biomarker can be nitric oxide. In some implementations, the functional coating can include an aptamer or an antibody.

In some implementations, the channel material between the drain electrode and the source electrode can have a length between 5 μm and 50 μm and a width between 25 μm and 125 μm. The device can include a microfluidic flow channel fluidically coupled with the fluid chamber. The microfluidic flow channel can include at least one separation region to remove undesirable particles from the fluid sample.

According to at least one aspect of the disclosure, a method can include providing a microfluidic device. The device can include a fluid chamber. The device can include a biomarker sensor at least partially disposed in a wall defining the fluid chamber. The sensor can include a drain electrode and a source electrode. The sensor can include a channel material electrically coupling the drain electrode and the source electrode. The sensor can include a functional coating disposed on a surface of the channel material. The functional coating can bind with a biomarker in the sample fluid and, responsive to the binding of the biomarker with the functional coating, change a conductivity of the channel material. The method can include flowing a fluid through the microfluidic device and at least partially over the biomarker sensor. The method can include measuring a conductivity through the channel material. The method can include determining an amount of the biomarker in the fluid based on the measured conductivity through the channel material.

In some implementations, the channel material can include poly(3,4-ethylenedioxythiophene) polystyrene sulfonate. The functional coating can include hemin. The biomarker can be nitric oxide. In some implementations, the functional coating can include an aptamer or an antibody.

In some implementations, the method can include applying at least one acoustic wave to the fluid flowing through the microfluidic device to drive a plurality of undesirable particles in the fluid toward an outlet of the microfluidic device. The undesirable particles can include toxins, bacteria, viruses, erythrocytes, leukocytes, or thrombocytes.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 illustrates a top view of an example system to detect biomarkers.

FIGS. 2A and 2B illustrate block diagrams of different embodiments of the organic electrochemical transistor sensor that can be used in the system illustrated in FIG. 1.

FIGS. 3A-3J illustrate cross-sectional views of a stackup during the fabrication of an example OECT sensor that can be used in the system illustrated in FIG. 1.

FIG. 4 illustrates a block diagram of an example method to detect biomarkers in a fluid sample.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

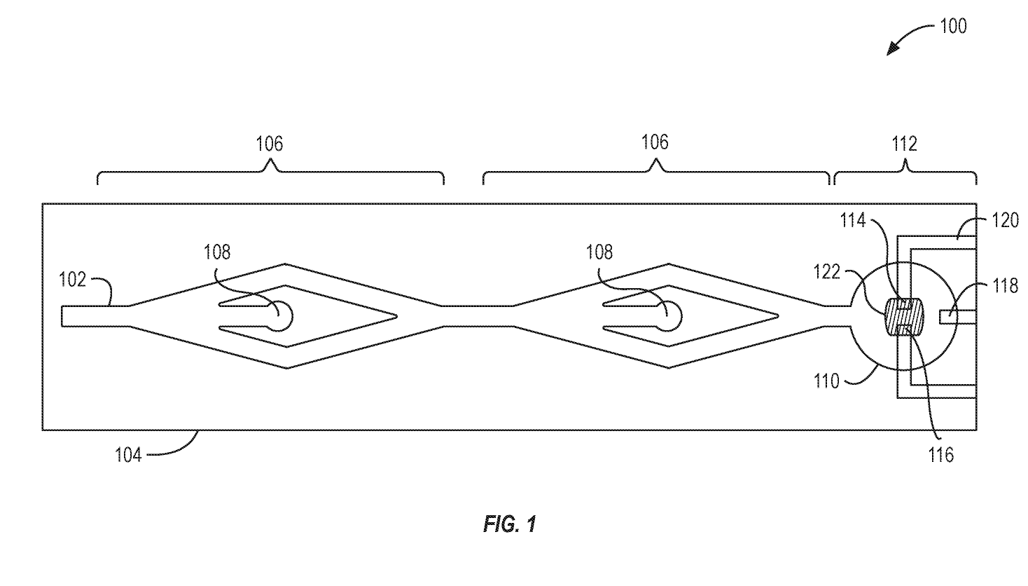

FIG. 1 illustrates a top view of an example system 100 to detect biomarkers. The system 100 can include a microfluidic flow channel 102 that is defined in a substrate 104. The microfluidic flow channel 102 can include multiple separation regions 106. Each of the separation regions 106 can include an outlet 108. The microfluidic flow channel 102 can terminate at a well 110. The well 110 can include an OECT sensor 112, which can also be referred to as a biomarker sensor 112. The OECT sensor 112 can include a drain electrode 114, a source electrode 116, and electrical traces 120. Each of the drain electrode 114, the source electrode 116, and the gate electrode 118 can be coupled with an electrical trace 120 to enable the respective electrode to be coupled with an external device. The OECT sensor 112 can include a channel material 122 to enable electrical contact between the drain electrode 114 and the source electrode 116. The components of the OECT sensor 112 can be components of a base layer on which the substrate 104 is positioned. For example, the base layer can be a printed circuit board (PCB).

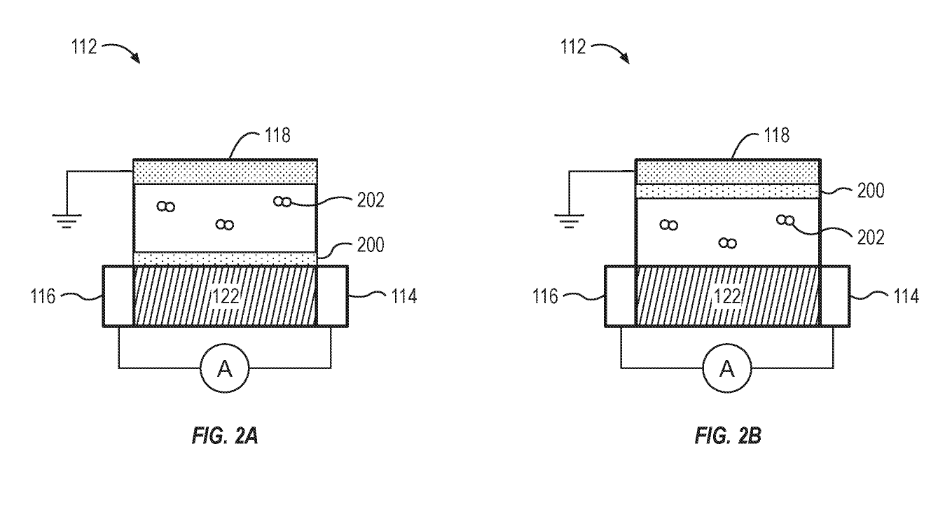

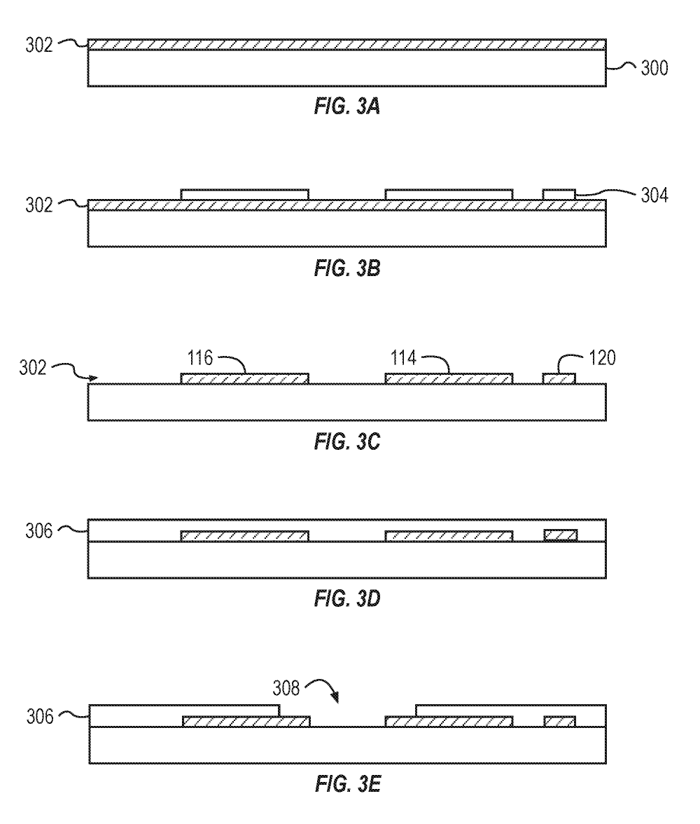

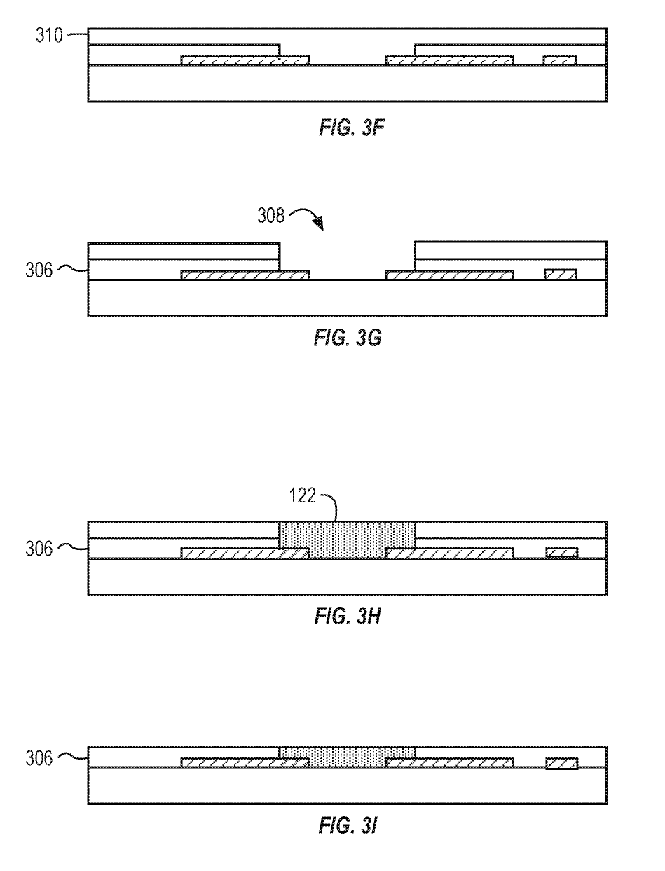

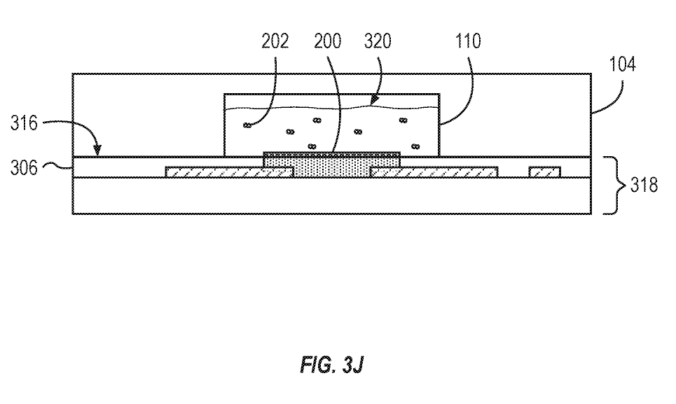

The system 100 can include a OECT sensor 112. The OECT sensor 112 is described further in relation to FIGS. 2A and 2B and FIGS. 3A-3J. The OECT sensor 112 is configured to detect biomarkers. The biomarker can be nitric oxide, which can be a biomarker for sepsis. The OECT sensor 112 can detect biomarkers such as cytokines, DNA, antibodies, antigens, protein, toxin, or other chemical. The OECT sensor 112 includes an organic electrochemical transistor. The OECT sensor 112 can include a channel material 122 that makes an electrical connection between the drain electrode 114 and the source electrode 116. For example, the channel material 122 between the drain electrode 114 and the source electrode 116 can be conductive. The channel material 122 can include a conductive polymer such as poly(3,4-ethylenedioxythiophene) polystyrene sulfonate (PEDOT:PSS).

The functionalized coating can change the conductivity of the channel material 122 in the presence of the biomarker. For detecting a biomarker such as nitric oxide, the functionalized coating can include Hemin (e.g., ferric chloride heme or C₃₄H₃₂ClFeN₄O₄) or Hemin chloride. The Hemin (or other functionalized coating) can functionalize the channel material 122 (e.g., the PEDOT:PSS) via non-covalent Pi-Pi bonding. For example, nitric oxide can transfer electrons to the hemin, which can change the conductivity of the channel material 122. The change in conductivity through the channel material 122 can be detected in real-time, via the electrical traces 122, by a current meter, for example. The magnitude of the current change indicates the amount of biomarker present in a test sample. For example, a relatively large change in the current through the OECT sensor 112 can indicate a relatively large amount of biomarker in the test sample. The OECT sensor 112, via the organic electrochemical transistor, can locally amplify an input signal before output and detection by the current meter.

The channel material 122 can have a length between about 5 μm and about 50 μm, between about 5 μm and about 25 μm, between about 5 μm and about 15 μm, or between about 10 μm and about 15 μm. The channel material 122 can have a width between about 25 μm and about 125 μm, between about 25 μm and about 100 μm, or between about 50 μm and about 100. The size of the gate electrode can have a length between about 50 μm and about 250 μm, between about 50 μm and about 200 μm, or between about 100 μm and about 200 μm. The size of the gate electrode can have a width between about 50 μm and about 250 μm, between about 50 μm and about 200 μm, or between about 100 μm and about 200 μm.

FIG. 1 illustrates the OECT sensor 112 in a microfluidic fluidic device. The OECT sensor 112 can be included in systems that have different configurations of microfluidic flow channels 102. For example, the microfluidic flow channel 102 could include additional or no separation regions 106. The OECT sensor 112 can be included in microfluidic systems that do not include a microfluidic flow channel 102. For example, the OECT sensors 112 can be included in the floor of each well of a multi-well plate. In some implementations, the OECT sensor 112 is not included in a microfluidic system. The OECT sensor 112 can be a component of an implantable device or immersible probe. For example, the OECT sensor 112 can be included in the probe of a biomarker detection device and that is configured to enable a user to immerse the probe into a sample.

The system 100 illustrated in FIG. 1 includes the microfluidic flow channel 102. A fluid sample can flow from an inlet of the microfluidic flow channel 102 toward the well 110 and OECT sensor 112. The fluid sample can include any fluid that is to be tested for biomarkers. The fluid sample can include blood, urine, saliva, sweat, or other bodily fluids. The bodily fluid can contain toxins, bacteria, viruses, cells, particles, or any combination thereof. For example, blood can include formed elements such as erythrocytes (e.g., red blood cells), leukocytes (e.g., white blood cells), thrombocytes (e.g., platelets); bacteria; viruses; toxins; or any combination thereof.

The microfluidic flow channel 102 can include one or more separation regions 106 that can remove undesirable particles or cells from the sample fluid via the outlets 108. For example, for a blood sample, the separation regions 106 can be configured remove bacteria and formed elements such that substantially only plasma, viruses, and biomarkers flow into the well 110 and interact with the OECT sensor 112. The separation regions 106 can remove undesirable particles that can interfere with the OECT sensor's ability to detect biomarkers. For example, nitric oxide can be a biomarker. However, the hemoglobin in red blood cells can act as a nitric oxide scavenger reducing the amount of free nitric oxide in the test sample. Removal of the red blood cells can preserve the level of free nitric oxide present in the test sample during the testing phase. The removal of particles, such as the red blood cells, enable a lower limit of detection (LOD) for the OECT sensor 112.

To remove particles from fluid flowing through the microfluidic flow channel 102, the system 100 can be coupled with one or more acoustic wave generators. For example, the system 100 can be coupled with a platform that positions an acoustic wave generator below each of the separation regions 106. The acoustic wave generators can impart a standing wave across the separation region 106. Particles (e.g., blood cells and bacteria cells) within the fluid sample can be driven towards the nodes or anti-nodes of the standing acoustic wave based the sign of the particles' contrast factor with respect to the fluid sample. For example, formed elements can have a positive contrast factor and can be driven, by the standing acoustic wave, towards the nodes of the standing acoustic wave. Particles with a negative contrast factor can be driven towards the antinode of the standing acoustic wave. The width of the microfluidic flow channel 102 prior to the separation regions 106 and the placement of the acoustic wave generators can be configured such that the standing acoustic wave forms a node or antinode near the central, longitudinal axis of the microfluidic flow channel 102.

The first separation region 106 can drive the formed elements towards the central, longitudinal axis of the microfluidic flow channel 102 (or other position of the standing acoustic wave's node) such that the formed elements exit the system 100 through the first outlet 108. The first separation region 106 can drive the other components of the fluid sample, for example, bacteria, plasma, and virus toward the walls of the microfluidic flow channel 102 such the components pass to the second separation region 106. In some implementations, particles other than the formed elements (e.g., the bacteria, plasma, and virus) can also be driven towards the central, longitudinal axis (and outlet 108), but at a rate slower than the formed elements. For these particles, the rate of movement towards the central, longitudinal axis may not be great enough to enable the particles to be sufficiently close to the central, longitudinal axis to exit through the first outlet 108 and the particles can pass to the second separation region 106.

The second separation region 106 can drive the remaining undesirable particles (e.g., bacteria) toward the central, longitudinal axis of the microfluidic flow channel 102 such that the remaining particles exit the microfluidic flow channel 102 through the second outlet 108. The remaining components of the fluid sample (e.g., the plasma, virus, and biomarkers) can flow into the well 110.

The microfluidic flow channel 102 can be formed within the substrate 104. The substrate 104 can include thermoplastics or other lossy plastics, such as, but not limited to, such as, polystyrene, acrylic (polymethylmethacrylate), polysulfone, polycarbonate, polyethylene, polypropylene, cyclic olefin copolymer, silicone, liquid crystal polymer, polyimide, polyetherimide, and polyvinylidene fluoride. The microfluidic flow channel 102 can be manufactured by a number of manufacturing techniques, including, but not limited to, milling, molding, embossing, and etching.

FIGS. 2A and 2B illustrate block diagrams of different embodiments of the OECT sensor 112 that can be used in the system 100. FIG. 2A illustrates an example OECT sensor 112 where the functionalized coating 200 (which can also be referred to as the coating 200) is applied to the channel material 122. FIG. 2B illustrates an example OECT sensor 112 where the coating 200 is applied to the gate electrode 118.

In FIGS. 2A and 2B, the gate electrode 118 is illustrated as above the drain electrode 114 and the source electrode 116. The biomarkers 202 can be in a sample fluid that is in contact with the gate electrode 118, the source electrode 116, and the drain electrode 114. In some implementations, the gate electrode 118 can be in the substantially the same plane as the drain electrode 114 and the source electrode 116. For example, as illustrated in FIG. 1, the drain electrode 114, the source electrode 116, and the gate electrode 118 can each be components of a base layer (e.g., PCB) on which the substrate 104 is coupled. The drain electrode 114, the source electrode 116, and the gate electrode 118 can be components of the floor of the well 110 and be exposed to the sample fluid in the well 110. The gate electrode 118 can be positioned in a plane different than that of the drain electrode 114 and the source electrode 116. For example, the drain electrode 114 and the source electrode 116 can be a component of the floor of the well 110 and the gate electrode 118 can be a component of a wall of the well 110.

The OECT sensor 112 includes the drain electrode 114, the source electrode 116, and the gate electrode 118. The drain electrode 114 and the source electrode 116 can be electrically coupled through the channel material 122. The drain electrode 114, the source electrode 116, the gate electrode 118, and the electrical traces 122 can include an electrically conductive metal such as gold, platinum, silver, or copper.

The channel material 122 can be a conductive polymer. The conductive polymer can include PEDOT:PSS. The channel material 122 can come into contact with both the drain electrode 114 and the source electrode 116 to form an electrochemical transistor. The channel material 122 can have a transconductance between about 2000 μs and about 2750 82 s, between about 2000 μs and about 3500 μs, between about 2000 μs and about 4250 μs, or between about 2000 μs and about 5000 μs. In some implementations, the channel material 122 can have a transconductance of about 4000 μs. The relatively high transconductance of the OECT sensor 112 can enable local amplification of an input signal before output and detection by a current or voltage meter. The OECT sensor 112 can have a footprint between about 20 μm×20 μm and about 100 μm×100 μm, between about 35 μm×35 μm and about 75 μm×75 μm, or between about 45 μm×45 μm and about 55 μm×55 μm.

In some implementations, the channel material 122 can be patterned, as described in relation to FIGS. 3A-3J, to fill a void between the drain electrode 114 and the source electrode 116. For example, the drain electrode 114 and the source electrode 116 can first be patterned onto a substrate. Using a mask, the channel material 122 can be patterned into the space between the drain electrode 114 and the source electrode 116. The channel material 122 can be patterned to cover at least a portion of the drain electrode 114 and the source electrode 116. In contact with at least a portion of the drain electrode 114 and the source electrode 116, the channel material 122 can form an electrical connection between the drain electrode 114 and the source electrode 116.

The OECT sensor 112 can include a coating 200 that covers at least a portion of the channel material 122. The coating 200 can be a functionalized coating with a bio-recognition element which interacts or bind to biomarkers. In FIG. 2A, the coating 200 is patterned over the channel material 122. An interaction of biomarker with bio-recognition element can cause a charge transfer, which can result in change in conductivity of the channel material 122. This change in conductivity can be measured as a change in source and drain current of the transistor. In FIG. 2B, the coating 200 is patterned over the gate electrode 118. The gate electrode 118 can be functionalized with a coating 200 that can include an aptamer or antibody. Binding of biomarkers with bio-recognition elements of the coating 200 can induce a change in the work function of the gate electrode 118, which can change the effective gate voltage.

The coating 200 can change the conductivity of the channel material 122 or the change the work function of the gate electrode 118 (depending on the placement of the coating 200) in the presence of the biomarker 202. The coating 200 can include Hemin. The coating 200 can include aptamers or antibodies. The Hemin (or other coating 200) can functionalize the channel material 122 (e.g., the PEDOT:PSS) via non-covalent Pi-Pi bonding. The biomarker 202 can transfer electrons to the coating 200, which can change the conductivity of the channel material 122. The change in conductivity through the channel material 122 can be detected in real-time, via the electrical traces 122, by a current meter, for example. In some implementations, the coating 200 can be applied to the gating electrode 118.

FIGS. 3A-3J illustrate cross-sectional views of a stackup during the fabrication of an example OECT sensor 112 that can be used in the system illustrated in FIG. 1. Multiple OECT sensors 112 can be fabricated on a single substrate. The multiple OECT sensors 112 can be diced into individual OECT sensors 112 or arrays of OECT sensors 112.

FIG. 3A illustrates that a metal layer 302 is deposited onto a base layer 300. The base layer 300 can be coupled to a via a sacrificial layer. The base layer 300 can be a plastic or glass substrate. The base layer 300 can include parylene, polyimide precursor, or silicone precursor. The metal layer 302 can be deposited on the base layer 300. The layers can be deposited through evapouration, sputtering, or printing

FIG. 3B illustrates the addition of a photoresist 304 to the metal layer 302. The photoresist 304 can be applied to the metal layer 302. After the application of a mask, portions of the photoresist 304 can be cured or developed by exposure to ultra-violet light or other forms of radiation. The non-cured portions of the photoresist 304 are removed to leave a patterned photoresist 304 atop the metal layer 302.

FIG. 3C illustrates a cross-section of the stackup with a patterned metal layer 302. The patterned metal layer 302 is formed by removing the portions of the metal layer 302 that are exposed (e.g., not covered) by the photoresist 304. The exposed portions of the metal later 302 can be removed through plasma or reactive ion etching.

The patterned metal layer 302 can form the drain electrode 114, the source electrode 116, the gate electrode 118, and the electrical traces 120. FIG. 3C illustrates a cross section through the drain electrode 114, the source electrode 116, and an electrical trace 120. The portion of the electrical trace 120 illustrated in FIG. 3C can, for example, electrically couple with the gate electrode 118.

FIG. 3D illustrates the addition of an insulating layer 306 to the stackup. The insulating layer 306 can be spin coated onto the stackup to a depth of between about 1 μm and about 5 μm or between about 2 μm and about 5 μm. The insulating layer 306 can encase the drain electrode 114, the source electrode 116, and the electrical trace 120. The insulating layer 306 can also encase other components of the metal layer 302, such as the gate electrode 118. The insulating layer 306 can include SU-8.

FIG. 3E illustrates the forming a void 308 in the insulating layer 306. The insulating layer 306 can be patterned to remove a portion of the insulating layer 306. The removed portions of the insulating layer 306 can expose at least a portion of the source electrode 116, the drain electrode 114, and the gate electrode 118.

FIG. 3F illustrates the patterning of a photoresist 310. The photoresist 310 can be spin coated onto the stackup. The photoresist material can fill the void 308 and can cover the insulating layer 306 and the exposed portions of the metal layer 302. FIG. 3G illustrated the reforming of the void 308. The photoresist 310 can be patterned to remove a portion of the photoresist 310 to reform the void 308.

FIG. 3H illustrated the patterning of the channel material 122. The channel material 122 can be spin coated onto the stack to fill the void 308. The channel material 122 can have a thickness between about 100 μm and about 500 μm, between about 100 μm and about 400 μm, or between about 100 μm and about 300 μm. The channel material 122 can include PEDOT:PSS.

FIG. 3I illustrates the formed stackup. The photoresist 310 and the excess channel material 122 can be removed by a life-off process by sonocatting in 3-propoanoal and Acetone 1:1 mix. In some implementations, the top surface of the stackup can be ground to provide a substantially planar top surface of the stackup. Once planarized, the channel material 122 can be functionalized. For example, the coating 200 can be applied to the channel material 122 or the gate electrode 118.

The stackup can be diced to form individual OECT sensors 112 or arrays of OECT sensors 112. The OECT sensors 112 can be released from the wafer by dissolving the sacrificial layer that coupled the stackup to the wafer. The diced stackup including the OECT sensor 112 or array of OECT sensors 112 can be used as a base layer for the system 100, illustrated in FIG. 1.

FIG. 3J, and also referring to FIG. 1, among others, illustrates the completed OECT sensor 112 formed in the steps illustrated in FIGS. 3A-3I. The OECT sensor 112 is a component of the base layer 318. In some implementations, the method of manufacture can include coupling the base layer 318 with the substrate 104. The microfluidic flow channel 102 and the well 110 can be machined or etched into a face 316 of the substrate 104. The face 316 of the substrate 104 can be coupled with the base layer 318. The base layer 318 can form the floor of the microfluidic flow channel 102 and the well 110 such that the coating 200 comes into contact with the fluid sample 320. The fluid sample 320 can include biomarkers 202.

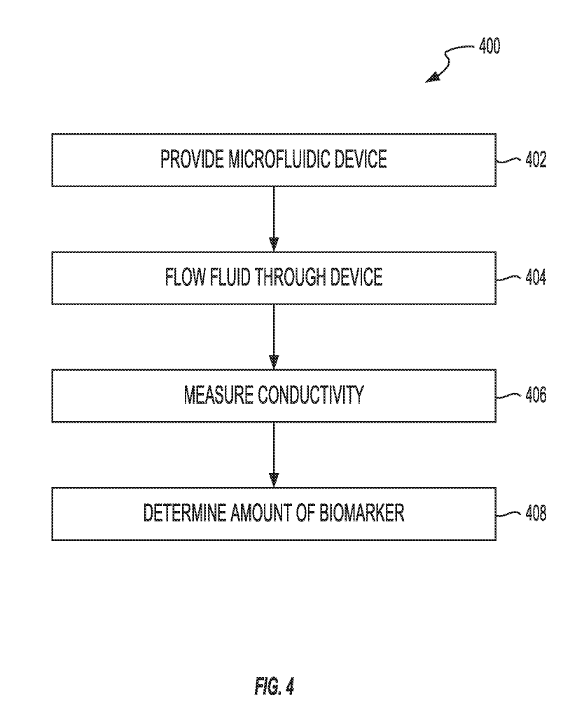

FIG. 4 illustrates a block diagram of an example method 400 to detect a biomarker. The method 400 can include providing a microfluidic device (BLOCK 402). The method 400 can include flowing a fluid through the device (BLOCK 404). The method 400 can include measuring a conductivity (BLOCK 406). The method 400 can include determining an amount of biomarker present in the fluid (BLOCK 408).

As set forth above, the method 400 can include providing a microfluidic device (BLOCK 402). The microfluidic device can be similar to any of the microfluidic devices described herein. For example, and referring to FIG. 1, among others, the microfluidic can include a microfluidic flow channel 102 that is defined in a substrate 104. The microfluidic flow channel 102 can include a well 110, which can be referred to as a fluid chamber. The microfluidic device can include a biomarker sensor. The biomarker sensor can be exposed to fluid flowing through the microfluidic flow channel 102 or fluid within the well 100. For example, the biomarker sensor can be at least partially disposed in a wall defining the well 110. The biomarker sensor can include a drain electrode, a source electrode, and a gate electrode. The biomarker sensor can include a channel material electrically coupling the drain electrode and the source electrode. The biomarker sensor can include a functional coating. The function coating can be disposed on a surface of the channel material, as illustrated in FIG. 2A, or on a surface of the gate electrode, as illustrated in FIG. 2B. The functional coating can bind or otherwise interact with a biomarker in the sample fluid. Responsive to binding or interacting with the biomarker, the functional coating can change the conductivity of the channel material or the effective gate voltage of the gate electrode.

The method 400 can include flowing a fluid through the microfluidic device (BLOCK 404). In some implementations, the fluid can be blood. The blood can include a biomarker, such as nitric oxide, that is to be measured with biomarker sensor. The blood can include one or more undesirable particles. The undesirable particles can include toxins, bacteria, viruses, erythrocytes, leukocytes, thrombocytes, or any combination thereof. In some implementations, the method can include filtering out or otherwise removing the undesirable particles from the fluid (e.g., the blood). For example, the microfluidic flow channel can include one or more separation regions. Within the separation regions, a standing acoustic wave can be applied to the fluid flowing through the microfluidic flow channel. The standing acoustic wave can drive the undesirable particles towards outlets of the microfluidic channel. Removing the undesirable particles, such as the red blood cells, can enable the biomarker sensor to have a lower limit of detection.

The method 400 can include measuring a conductivity (BLOCK 406). As the fluid flows over (or otherwise immerses) the functional coating, The functional coating can bind with a biomarker in the sample fluid and, responsive to the binding of the biomarker with the functional coating, change a conductivity of the channel material. For detecting a biomarker such as nitric oxide, the functionalized coating can include Hemin (e.g., ferric chloride heme or C₃₄H₃₂ClFeN₄O₄) or Hemin chloride. The Hemin (or other functionalized coating) can functionalize the channel material 122 via non-covalent Pi-Pi bonding. For example, nitric oxide can transfer electrons to the hemin, which can change the conductivity of the channel material. The change in conductivity through the channel material can be detected in real-time, via the electrical traces, by a current meter, for example.

The method 400 can include determining an amount of biomarker (BLOCK 408). The determined amount of biomarker in the fluid sample can be based on the change in conductivity of the channel material. For example, the conductivity of the channel material can be measured or otherwise known when the biomarker sensor is exposed to a fluid that does not contain the biomarker. The magnitude of the current change can indicate the amount of biomarker present in the fluid sample. For example, a relatively large change in the current through the biomarker sensor can indicate a relatively large amount of biomarker in the fluid sample. The biomarker sensor, via the organic electrochemical transistor, can locally amplify an input signal before output and detection by the current meter. In some implementations, the relationship between the change in conductivity and the amount of biomarker in the fluid sample can be linear, enabling a conversion between the amount of conductivity change to the amount of biomarker in the fluid sample.

While operations are depicted in the drawings in a particular order, such operations are not required to be performed in the particular order shown or in sequential order, and all illustrated operations are not required to be performed. Actions described herein can be performed in a different order.

The separation of various system components does not require separation in all implementations, and the described program components can be included in a single hardware or software product.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the term “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence has any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed:

1. A microfluidic device, comprising:
a fluid chamber to hold a sample fluid; and
a biomarker sensor at least partially disposed in a wall defining the fluid chamber; the biomarker sensor comprising:
a drain electrode;
a source electrode;
a channel material electrically coupling the drain electrode and the source electrode; and
a functional coating disposed on a surface of the channel material, the functional coating configured to bind with a biomarker in the sample fluid and, responsive to the binding of the biomarker with the functional coating, change a conductivity of the channel material.

2. The device of claim 1, wherein the channel material comprises poly(3,4-ethylenedioxythiophene) polystyrene sulfonate.

3. The device of claim 1, wherein the functional coating comprises hemin.

4. The device of claim 3, wherein the biomarker is nitric oxide.

5. The device of claim 1, wherein the functional coating comprises an aptamer or an antibody.

6. The device of claim 1, wherein the channel material between the drain electrode and the source electrode has a length between 5 μm and 50 μm and a width between 25 μm and 125 μm.

7. The device of claim 1, further comprising a microfluidic flow channel fluidically coupled with the fluid chamber, the microfluidic flow channel comprising at least one separation region to remove undesirable particles from the fluid sample.

8. A microfluidic device, comprising:
a fluid chamber to hold a sample fluid; and
a biomarker sensor at least partially disposed in a wall defining the fluid chamber; the biomarker sensor comprising:
a drain electrode;
a source electrode;
a channel material electrically coupling the drain electrode and the source electrode;
a gate electrode; and
a functional coating disposed on a surface of the gate electrode, the functional coating configured to bind with a biomarker in the sample fluid and, responsive to the binding of the biomarker with the functional coating, change a gate voltage of the gate electrode.

9. The device of claim 8, wherein the channel material comprises poly(3,4-ethylenedioxythiophene) polystyrene sulfonate.

10. The device of claim 8, wherein the functional coating comprises hemin.

11. The device of claim 10, wherein the biomarker is nitric oxide.

12. The device of claim 8, wherein the functional coating comprises an aptamer or an antibody.

13. The device of claim 8, further comprising a microfluidic flow channel fluidically coupled with the fluid chamber, the microfluidic flow channel comprising at least one separation region to remove undesirable particles from the fluid sample

14. A method, comprising:
providing a microfluidic device comprising:
a fluid chamber; and
a biomarker sensor at least partially disposed in a wall defining the fluid chamber; the biomarker sensor comprising:
a drain electrode;
a source electrode;
a channel material electrically coupling the drain electrode and the source electrode; and
a functional coating disposed on a surface of the channel material, the functional coating configured to bind with a biomarker in the sample fluid and, responsive to the binding of the biomarker with the functional coating, change a conductivity of the channel material;
flowing a fluid through the microfluidic device and at least partially over the biomarker sensor;
measuring a conductivity through the channel material; and
determining an amount of the biomarker in the fluid based on the measured conductivity through the channel material.

15. The method of claim 14, wherein the channel material comprises poly(3,4-ethylenedioxythiophene) polystyrene sulfonate.

16. The method of claim 14,wherein the functional coating comprises hemin.

17. The method of claim 16, wherein the biomarker is nitric oxide.

18. The method of claim 14,wherein the functional coating comprises an aptamer or an antibody.

19. The method of claim 14, further comprising applying at least one acoustic wave to the fluid flowing through the microfluidic device to drive a plurality of undesirable particles in the fluid toward an outlet of the microfluidic device.

20. The method of claim 19, wherein the undesirable particles comprise toxins, bacteria, viruses, erythrocytes, leukocytes, or thrombocytes.
